(19) United States
(12) Patent Application Publication
(10) Pub. No.: US 2004/0102784 A1
(43) Pub. Date: May 27, 2004

(54) Curettage instrument

(76) Inventors: Denis Pasquet, Pessac (FR); Regis Le Couedic, Bordeaux (FR)

Correspondence Address: MERCHANT & GOULD PC, P.O. BOX 2903, MINNEAPOLIS, MN, 55402-0903, US

(21) Appl. No.: 10/332,374

(22) PCT Filed: Jul. 12, 2001
(86) PCT No.: PCT/FR01/02259

(30) Foreign Application Priority Data
Jul. 12, 2000 (FR) 00 09095

(51) Int. Cl. A61B 17/00

(52) U.S. Cl. 606/84

(57) ABSTRACT

The invention concerns a curettage instrument for scraping the intervertebral space and in particular the vertebral end-plates, comprising a first part (10) forming a handle and a second active part (12). The second active part (12), elongated in shape along a main axis (A), having a first end (14) linked to the first part (10) and a second free end (16), comprises at least a longitudinal portion (18) forming a cutting edge, the longitudinal portion (18) extending from the first end (14) of the second part (12) towards the second free end (16) of the second part (12) and has a median portion. The distance separating said median portion from the longitudinal portion (18) of the main axis (A) is greater than the distance separating each of the ends of the longitudinal portion (18) of the main axis (A), whereby when the second part (12) is driven in rotation against the surface of the vertebral end-plate, this causes a cavity to be formed whereof the maximum depth corresponds to said distance separating said median part from the main axis (A).

The present invention relates to a curettage tool for curetting the intervertebral space, and it relates more particularly to a tool designed to revivify the vertebral plates.

The invention is particularly applicable to surgery of the intervertebral disk in which it is necessary, in some circumstances, for the damaged intervertebral disk to be completely extracted in order to replace it with a prosthetic element.

For some therapeutic indications, treatment of degenerative disks consists, in particular, in replacing the intervertebral disk with a disk prosthesis or with bone grafts that are immobilized in an intersomatic cage. Operations are carried out when the intervertebral disk can no longer perform its function. The intervertebral disk must withstand the transmission of the forces of gravity while maintaining determined intervertebral spacing, and while also enabling ample movements between the vertebrae in all directions.

After dissecting and exposing the damaged intervertebral disk, the surgical act itself requires the disk to be excised with a scalpel and removed in its entirety. This is made easier by moving the adjacent vertebral bodies apart from each other.

When treatment requires a graft to be inserted between the vertebrae, the vertebral plates must be revivified so that they bleed in order to enable the graft to take. This operation is generally performed by means of a curette which scrapes the vertebral plates. However, that tool produces grooves on the wall of the vertebral plates and does not make it possible to revivify evenly. In addition, since the vertebral plates are concave in shape, it is not easy to scrape them in order to obtain an even surface.

An object of the present invention is to propose a curettage tool that enables the vertebral plates to be revivified in the intervertebral space while complying with the concave shape of the plates, and more generally the biconcave shape of the intervertebral space.

To achieve this object the invention provides a curettage tool designed to scrape an intervertebral space and in particular to scrape vertebral plates, the tool comprising a handle-forming first portion and an active second portion, said active second portion is of elongate shape along a main axis, presenting a first end connected to said first portion and a second end that is free, and comprises at least one longitudinal portion forming a scraper blade, said longitudinal portion extending from said first end of said second portion to said free second end of said second portion and presenting a middle portion, the distance between said middle portion of said longitudinal portion and said main axis being greater than the distance between each of the ends of said longitudinal portion and said main axis whereby rotating said second portion against the surface of said intervertebral plate causes a cavity to be hollowed out having a maximum depth that corresponds to said distance between said middle portion and said main axis.

Thus, a characteristic of the curettage tool resides in the shape of said scraper-forming longitudinal portion which extends from said first end to said free second end of said second portion and defines a curve whose distance from the main axis is at a maximum in its middle portion. As a result, rotating said active second portion about its main axis causes the scraper-forming longitudinal portion to rotate and enables the tangential surface against which said second portion is applied to be scraped perpendicular to its main axis. It will be understood that when the relative positions of said surface and said main axis are fixed, the surface that is scraped depends on the radii of curvature of the path followed by said scraper and of said tangential surface. However, the radius of curvature of said path is such that the scraped surface is relatively plane compared with the surfaces obtained by means of prior art tools, Furthermore, since said scraper presents a maximum, the cutting line matches the concave shape of the vertebral plates.

According to an advantageous characteristic, the active second portion presenting an elongate shape is a solid of revolution defined by rotating a generator line curve about said main axis, said curve including a maximum relative to said main axis.

As a result, the shape of the active second portion matches the shape of the biconcave intervertebral space whatever the angular position of said second portion since it is cylindrically symmetrical.

According to another advantageous characteristic, the generator line presents a radius of curvature that varies continuously so as to obtain a relatively even surface state of the vertebral plates.

In order to cover the entire intervertebral space, which is substantially semi-circular and which is downwardly and upwardly defined by two substantially parallel vertebral plates, the active second portion of the tool must be rotated and displaced sideways.

The mean line of said scraper-forming longitudinal portion, directed in the travel direction of said portion and contained in a plane perpendicular to said main axis, preferably forms an angle with a radial half line, directed away from said main axis towards said longitudinal portion and contained in said perpendicular plane, which angle is less than 90°.

Thus, according to this characteristic, the angle of said scraper-forming portion with the surface of the intervertebral plate tangential to the path of said portion is less than 90° thereby enabling an optimum scraping effect to be obtained when the tool is rotated.

In a particular embodiment said scraper-forming longitudinal portion is formed by at least one projecting edge of an oblong recess formed in the wall of said second portion of said tool.

Thus, the projecting edge of the oblong recess formed in the active second portion forms an edge which can be machined in such a manner as to obtain a scraper.

Advantageously, said second portion presents an axial bore along the main axis, and the bottom of said recess opens out to said bore. This characteristic enables the shavings produced from scraping the surface of the vertebral plates to be evacuated since, while scraping is being performed, they pass through the bottom of the recess and are evacuated to the inside of the bore. When the bore space becomes full of shavings during use of the tool, it is necessary to remove said tool from the intervertebral space in order to remove the shavings.

In another particular embodiment, said oblong recess is formed in said second portion on a mid plane that does not contain said main axis so as to form said projecting edge. As a result, the scraper is formed easily without special machining of the edge of the oblong recess. By forming the oblong recess on a mid plane that is inclined relative to the radial axis, an edge is obtained which is sufficiently sharp to form the scraper.

In a particular implementation of the invention, said oblong recess is formed around said second portion so as to form a helix along the main axis. Thus, when said active second portion is rotated, the cutting line of the scraper-forming longitudinal portion is oblique relative to the surface of the vertebral plate to be scraped, thereby enabling a better cut.

In another particular implementation of the invention, said second portion presents a plurality of scraper-forming longitudinal portions that are substantially parallel to each other. As a result, the active second portion of the tool, inserted between the two vertebral plates, is suitable for scraping both the upper plate and the lower plate simultaneously, providing the dimensions of said active second portion correspond to the intervertebral dimensions.

Other characteristics and advantages of the invention will appear on reading the following description of particular embodiments of the invention given by way of non-limiting indication with reference to the accompanying drawings, in which:

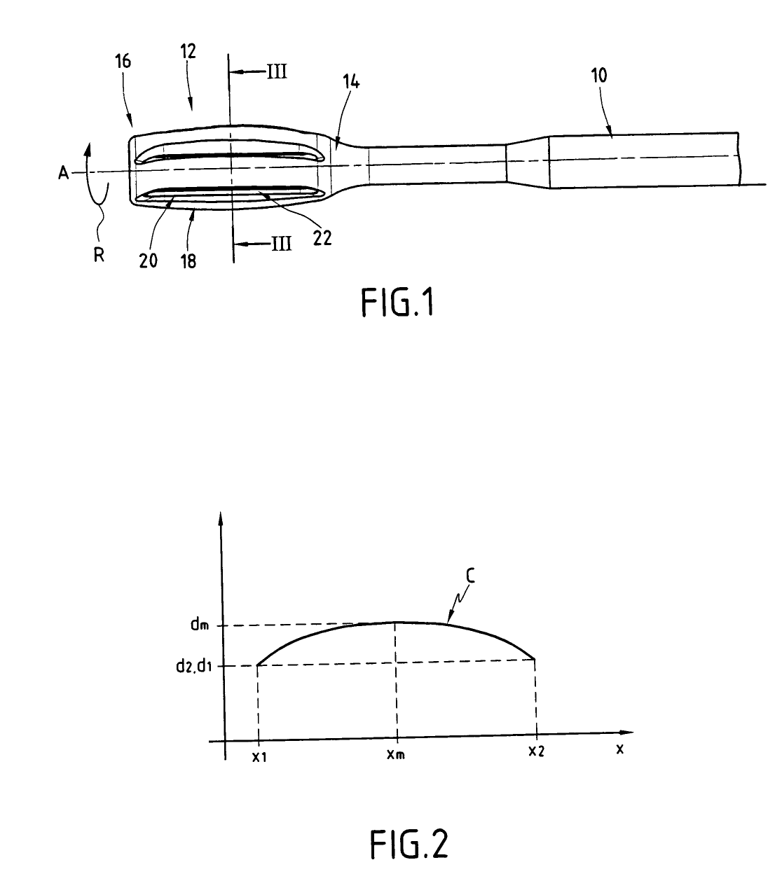

FIG. 1 is a diagrammatic fragmentary view in elevation of a curettage tool of the invention;

FIG. 2 is a graph showing a portion of curve in a (0, x, y) frame of reference, suitable for defining a solid of revolution;

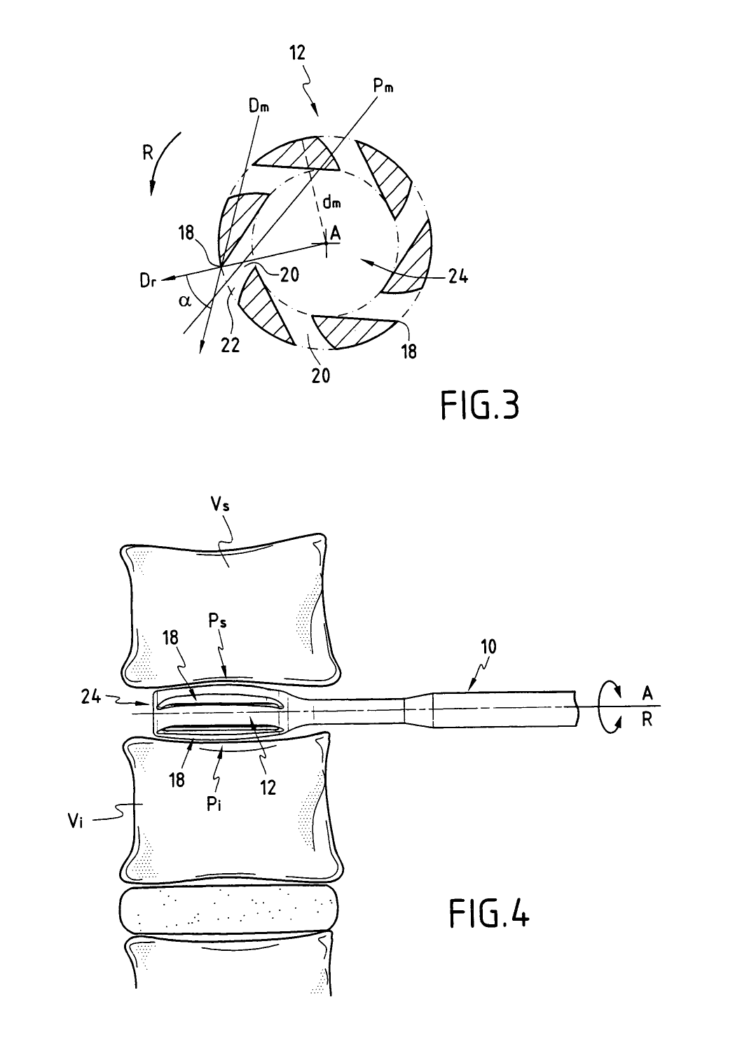

FIG. 3 is a cross-section of the active second portion of the invention, taken at right angles to the middle of the longitudinal portion shown in FIG. 1; and

FIG. 4 is a diagrammatic view showing the curettage tool being used in the intervertebral space.

With reference firstly to FIGS. 1 and 2, the curettage tool is described in general manner.

In FIG. 1, the tool which presents cylindrical symmetry is shown in elevation and includes a handle-forming first portion 10 that is axially extended by an active second portion 12; the axis common to the two portions is called the main axis A. The active second portion 12 presents a first end 14 connected to the handle-forming portion 10, and a free second end 16. The active second portion 12 comprises, at it periphery, a longitudinal portion 18 forming a scraper blade, said longitudinal portion extending from its first end 14 to its free second end 16.

The longitudinal portion 18 presents a concave shape whose concave side faces towards the main axis A. With reference to FIG. 2, the shape of the active second portion 12 is described in general manner, and the shape of the longitudinal portion 18 is described in detailed manner.

FIG. 2 shows a fraction of a curve C presenting a maximum of X and Y co-ordinates xm and ym in its middle portion, and two ends of X co-ordinates x1 and x2 respectively, and of Y co-ordinates d1 and d2 respectively that are substantially identical. The X-axis is comparable to the main axis A, and the active second portion 12 is defined by a solid of revolution obtained by rotating the curve fraction forming the generator line about the X-axis.

The curve fraction C presents continuous variation of its radius of curvature, which is at a maximum in its middle portion and which diminishes progressively towards the X-axis ends x1 and x2.

In a particular embodiment, the curve C is symmetrical about an axis intersecting the X-axis perpendicularly at xm.

As shown in FIG. 1, at least one oblong recess 20 is formed in the active second portion 12, said recess extending from the first end 14 to the free second end 16. The recess thus defines two opposite edges 18, 22, of which one 18 is capable of being transformed into a scraper-blade.

In view of the particular shape of the active second portion 12 defined by said solid of revolution, the edges 18 and 22 present a shape which matches that of the curve portion C. Thus the middle portion of the scraping edge forming the scraper-blade 18 presents a maximum corresponding to the maximum of the curve C.

In order to describe a particular embodiment of the invention, reference is now made to FIG. 3, corresponding to a cross-section through the middle of the active second portion 12.

FIG. 3 shows the active second portion 12 which presents a central bore 24 into which there open out six oblong recesses that are distributed around the periphery of said active second portion 12. FIG. 3 also shows the scraper-forming longitudinal portion 18 and the opposite edge 22 of the oblong recess 20.

The oblong recesses 20 formed longitudinally in the wall of the active second portion on a mid plane Pm are shown in section in FIG. 3. The mid plane Pm does not contain the axis A and it is sufficiently oblique relative to the wall of the active second portion 12 to constitute a projecting edge forming a scraper blade. Thus, the directed mean line Dm of the scraper-forming portion and the directed radial half line Dr form an angle α that is less than 90°.

It will be understood that rotation of the active second portion 12 of the tool in the counter-clockwise direction R causes the six scraper-forming projecting edges 18 to be displaced. Thus, two tangential surfaces at two diametrally opposite points of the active second portion 12 are scraped simultaneously by the scraper-forming portion 18.

Reference is now made to FIG. 4 in order to describe the conditions under which the tool of the invention is used. FIG. 4 shows the curettage tool as described above with reference to FIG. 1, interposed between two adjacent vertebrae Vs and Vi.

Once the defective intervertebral disk has been partly or completely removed, the active second portion 12 of the curettage tool is inserted between the vertebral plates Ps and Pi of the two vertebrae Vs and Vi, said vertebral plates defining a biconcave intervertebral space. The shape of the active second portion 12 of the curettage tool corresponds to the biconcave shape of the intervertebral space such that the wall of the upper vertebral plate Ps is tangential to the active portion 12, and such that the wall of the lower vertebral plate Pi is tangential to the active portion 12 at a diametrally opposite point.

In this configuration, rotating the first scraper-forming portion 10 in the counter-clockwise direction R, while keeping the tool axis A fixed, causes the scraper-forming longitudinal portions 18 to move against the vertebral plates Ps and Pi. As a result, the cutting line of said scraper scrapes the plates Ps and Pi and the shavings generated by the scraper-forming longitudinal portions penetrate into the bore 24.

It will be understood that the scrapers form longitudinal scrape marks in the walls of the vertebral plates Ps and Pi, said marks being of a width that depends on the radius of the active second portion 12, which is determined by the available intervertebral space.

In order to scrape the entire wall of the vertebral plates, the curettage tool must be displaced sideways while it is being rotated. The vertebral plates must be scraped until they bleed so that the bone graft which is to be inserted between the two plates can take hold.

In a particular embodiment, the oblong recesses are formed around the active second portion so as to form a helix along the main axis A. In some circumstances, this disposition can improve scraping of the wall of the vertebral plates since the scraper-forming portion is oblique relative to the wall and the cutting line tends to slide into the already scraped portion.

The curettage tool of the invention can be made of stainless steel or of any other solid material.

1/ A curettage tool designed to scrape an intervertebral space and in particular to scrape vertebral plates, the tool comprising a handle-forming first portion (10) and an active second portion (12),
the tool being characterized in that said active second portion (12) forms a solid of revolution defined by rotating a generator line curve (C) about a main axis (A) coinciding with the axis of the handle, said generator line curve having a distance maximum (dm) away from said main axis (A), said active second portion (12), that is of elongate shape along said main axis (A) between a first end (14) connected to said first portion (10) and a second end (16) that is free, comprises a plurality of longitudinal portions (18) forming scraper blades angularly offset about said main axis (A), said longitudinal portion (18) extending from said first end (14) of said second portion (12) to said free second end (16) of said second portion (12) and presenting a middle portion,
and in that the distance (dm) between said middle portion of said longitudinal portion (18) and said main axis (A) is greater than the distance (d1, d2) between each of the ends of said longitudinal portion (18) and said main axis (A) whereby rotating said second portion (12) against the surface of said intervertebral plate causes a cavity to be hollowed out having a maximum depth that corresponds to said distance (dm) between said middle portion and said main axis (A).

2/ A curettage tool according to claim 1, characterized in that said generator line (C) presents a radius of curvature that varies continuously.

3/ A curettage tool according to claim 1 or claim 2, characterized in that the mean line (Dm) of said scraper-forming longitudinal portion (18), directed in the travel direction of said portion and contained in a plane perpendicular to said main axis (A), forms an angle (α) with a radial half line (Dr), directed away from said main axis (A) towards said longitudinal portion (18) and contained in said perpendicular plane, which angle is less than 90°.

4/ A curettage tool according to any one of claims 1 to 3, characterized in that said scraper-forming longitudinal portion (18) is formed by at least one projecting edge (18) of an oblong recess (20) formed in the wall of said second portion (12) of said tool.

5/ A curettage tool according to claim 4, characterized in that said second portion (12) presents an axial bore (24) along the main axis (A), and in that the bottom of said recess (20) opens out to said bore (24).

6/ A curettage tool according to claim 4 or claim 5, characterized in that said oblong recess (20) is formed in said second portion (12) on a mid plane (Pm) that does not contain said main axis (A) so as to form said projecting edge (18).

7/ A curettage tool according to any one of claims 4 to 6, characterized in that said oblong recess (20) is formed around said second portion (12) so as to form a helix along the main axis (A).

8/ A curettage tool according to any one of claims 1 to 7, characterized in that said second portion (12) presents a plurality of scraper-forming longitudinal portions (18) that are substantially parallel to each other.

9/ A curettage tool according to any one of claims 1 to 7, characterized in that said second portion (12) presents six scraper-forming longitudinal portions (18) that are substantially parallel to each other.
